(19) United States
(12) Patent Application Publication
(10) Pub. No.: US 2014/0163624 A1
(43) Pub. Date: Jun. 12, 2014

(54) Bone Screw with Deflectable Portion

(71) Applicants: Tzony Siegal, Moshav Shoeva (IL); Oded Loebl, Tel Mond (IL); Didier Toubia, Raanana (IL)

(72) Inventors: Tzony Siegal, Moshav Shoeva (IL); Oded Loebl, Tel Mond (IL); Didier Toubia, Raanana (IL)

(73) Assignee: NLT-SPINE LTD., Kfar Saba (IL)

(21) Appl. No.: 13/579,285

(22) PCT Filed: Aug. 2, 2012
(86) PCT No.: PCT/IB12/53973

§ 371 Date: Aug. 16, 2012

Related U.S. Application Data
(60) Provisional application No. 61/514,068, filed on Aug. 2, 2011.

(51) Int. Cl. A61B 17/86

(52) U.S. Cl. CPC A61B 17/864
USPC 606/304

(57) ABSTRACT

An anchoring element, such as a bone screw, may comprise an at least partially threaded shaft having a core and a longitudinal channel. An elongated element movable with the channel may be a tensioning element, for example affixed to the distal portion, and/or a rigid element. The core may have one or more lateral cut-outs, which may be V-shaped, at the distal portion. Each cut-out may be backed by an effective hinge that may be displaced from the longitudinal axis, and may have a transverse slit between the hinge and an opposite surface of the core from the cut-out. Upon axial movement of the elongated element, e.g. toward a proximal end of the shaft, the distal portion may deflect to an angle from the shaft's longitudinal axis. Alternatively, the anchoring element may be pre-biased to deflect and maintained straight by a rigid element until axial movement of the rigid element.

### FIELD AND BACKGROUND OF THE INVENTION

The present invention generally relates to apparatus and methods for anchoring elements such as screws and, more particularly, to apparatus and methods for a screw, such as a bone screw, with a deflectable portion to increase the pullout resistance of the screw and its resistance to loosening. The anchoring elements of the present invention may also be useful for other medical and non-medical applications.

The elderly are increasing their share in the overall number of orthopedic and particularly spinal surgical procedures. As incidence of osteoporosis increases with age it presents a challenge when pedicle screw fixation is considered.

Conventional screws have a given pullout strength. In a conventional bone screw, the advancing screw displaces trabecular bone, and the displaced spongy bone forms a thickened bony envelope around the shaft of the positioned screw. This condensed spongy bone concentration around the screw shaft is forced between the coils of the thread. The pull out strength depends on this concentration of the bone tissue on one hand, and on the resisting surface of the helical thread on the other.

The thread of the screw projecting from its core is a cumulative surface which faces and resists forces of pullout.

There is a compelling need for a screw (or other anchoring element) design and method that improves the pullout resistance of the screw, reduces the risk of undesired screw loosening and overcomes other disadvantages of the prior art.

### SUMMARY OF THE PRESENT INVENTION

One aspect of the present invention is an anchoring element, comprising a shaft having a core and a longitudinal channel, the shaft also having a distal portion and having threading along at least a portion of a length of the shaft; an elongated element movable within the channel, the elongated element being (i) a tensioning element or (ii) a rigid element or (iii) a tensioning element and a rigid element; one or more lateral cut-outs in the core at the distal portion or at a central portion of the shaft, each cut-out backed by an effective hinge, the channel traversing at least one side of one of the cut-outs, the elongated element and distal portion configured such that upon axial movement of the elongated element, the distal portion deflects to an angle from a longitudinal axis of the shaft.

A further aspect of the present invention is a method of inserting an anchoring element into an object, comprising taking an anchoring element, the anchoring element configured with a shaft that has a core and a longitudinal channel, the shaft also having a distal portion and having threading along at least a portion of a length of the shaft, the anchoring element having an elongated element configured to move within the channel, the core configured with one or more lateral cut-outs at the distal portion or at a central portion of the shaft such that each cut-out is backed by an effective hinge and such that the channel traverses at least one side of one of the cut-outs, inserting the anchoring element into the object; and moving the elongated element axially so that the distal portion deflects away from a longitudinal axis of the shaft.

A still further aspect of the present invention is a method of inserting an anchoring element into an object, comprising taking an anchoring element that is pre-biased to assume a deflected form such that a distal portion of a shaft of the anchoring element is at an angle to a longitudinal axis of the shaft, the shaft also having threading along at least a portion of a length of the shaft, the anchoring element configuring with a longitudinal channel housing a rigid elongated reinforcing element configured to move within the channel, the core having one or more lateral cut-outs at the distal portion or at a central portion of the shaft, each cut-put backed by an effective hinge, the channel traversing at least one side of one of the cut-outs; inserting the anchoring element into the object with the longitudinal channel housing a rigid elongated reinforcing element that temporarily holds the anchoring element in a straightened configuration; and moving the rigid elongated reinforcing element axially through the channel sufficient to release the anchoring element to the pre-biased deflected form.

A yet still further aspect of the present invention is a method of inserting an anchoring element into an object, comprising taking an anchoring element, the anchoring element configured with a shaft that has a core and a longitudinal channel, the shaft also having a distal portion and having threading along at least a portion of a length of the shaft, the anchoring element having an elongated element configured to move within the channel, the core configured with one or more lateral slits at the distal portion or at a central portion of the shaft such that each lateral slit is backed by an effective hinge and such that the channel traverses at least one of the slits, inserting the anchoring element into the object; and moving the elongated element axially toward a distal tip of the shaft so that the slits open and the distal portion deflects away from a longitudinal axis of the shaft.

A still further aspect of the present invention is an anchoring element, comprising a shaft having a core and a longitudinal channel, the shaft also having a distal portion and having threading along at least a portion of a length of the shaft; an elongated element movable within the channel, the elongated element being flexible enough to bend during deflection but sufficiently rigid to effectuate the deflection; one or more lateral cut-outs in the core at the distal portion or at a central portion of the shaft, each cut-out backed by an effective hinge, the channel traversing at least one side of one of the cut-outs, the elongated element and distal portion configured such that upon axial movement of the elongated element, the distal portion deflects to an angle from a longitudinal axis of the shaft.

These and other features, aspects and advantages of the present invention will become better understood with reference to the following drawings, descriptions and claims.

### BRIEF DESCRIPTION OF THE DRAWINGS

Various embodiments are herein described, by way of example only, with reference to the accompanying drawings, wherein:

FIG. 1 is a schematic illustration of a screw inserted into a pedicle 17, in accordance with one embodiment of the present invention;

FIG. 2 is a side view of a distal end 28 of a screw showing cut-outs, integral joints and relief slits, in accordance with one embodiment of the present invention;

FIG. 3A is an isometric view of a screw, in accordance with one embodiment of the present invention;

FIG. 3B is a side view of the screw of FIG. 3A, in accordance with one embodiment of the present invention;

FIG. 3C is a top view of the screw of FIG. 3A, in accordance with one embodiment of the present invention;

FIG. 3D is a vertical cross-sectional view of the screw of FIG. 3A, in accordance with one embodiment of the present invention;

FIG. 4A is a side view of the screw of FIG. 3A, opposite to the side depicted in FIG. 3B, shown in straight configuration of its distal tip, in accordance with one embodiment of the present invention;

FIG. 4B is a side view of a screw similar to FIG. 4A, except shown with the distal tip deflected, in accordance with one embodiment of the present invention;

FIG. 5 is an enlarged view of the distal tip in deflected position showing cut-out wedges that have closed together, showing open fissures, and also showing the tip in a straight position, in accordance with one embodiment of the present invention;

FIG. 6 is a schematic illustration of a screw inserted into a pedicle showing the lack of cyclic loading on the deflected tip of the screw adjacent the void, in accordance with one embodiment of the present invention;

FIG. 7A is a schematic view of a screw whose distal end is subdivided into two elongated segments, in accordance with one embodiment of the present invention;

FIG. 7B is a schematic view of the screw of FIG. 7A where the two elongated segments are deflected in different directions, in accordance with one embodiment of the present invention;

FIG. 7C is a schematic drawing showing a shaft with a single cut-out 33, in accordance with one embodiment of the present invention;

FIG. 8 is a flow chart of a method, in accordance with one embodiment of the present invention;

FIG. 9 is a flow chart of a further method, in accordance with one embodiment of the present invention;

FIG. 10A is a side view of an anchoring element in a straight configuration showing an elongated tensioning element, in accordance with one embodiment of the present invention;

FIG. 10B is a side view of the anchoring element of FIG. 10A in a deflected configuration showing the elongated tensioning element, in accordance with one embodiment of the present invention;

FIG. 10C is an isometric view of threaded shaft having a distal portion in deflected configuration, in accordance with one embodiment of the present invention;

FIG. 10D, 10E, 10F, 10G are sectional views taken along line A-A of FIG. 10C showing various alternatives of the cross-sectional shape of the longitudinal channel 32 or multiple channels 32, in accordance with one embodiment of the present invention;

FIG. 11A is an isometric view of a screw with a rigid reinforcing element in a peripheral channel in straight configuration, in accordance with one embodiment of the present invention;

FIG. 11B is an isometric view of the screw of FIG. 1A in deflected configuration, in accordance with one embodiment of the present invention;

FIG. 12A is a side view of a screw with a rigid reinforcing element in a central channel in straight configuration, in accordance with one embodiment of the present invention;

FIG. 12B is a side view of the screw of FIG. 11A in deflected configuration, in accordance with one embodiment of the present invention;

FIG. 13A is an isometric view of a screw with a partial thread on a proximal portion of the shaft, in accordance with one embodiment of the present invention;

FIG. 13B is an isometric view of a screw with a partial thread on a distal portion of the shaft, in accordance with one embodiment of the present invention;

FIG. 13C1 and FIG. 13C2 isometric views of a locking mechanism for maintaining deflection of the distal portion, in accordance with one embodiment of the present invention;

FIG. 14A is an isometric view of a screw with cut-outs on alternating sides of the core and in a straightened configuration, in accordance with one embodiment of the present invention;

FIG. 14B is an isometric view of a screw of FIG. 14A in a deflected form with the deflection in alternating directions, in accordance with one embodiment of the present invention;

FIG. 15A and FIG. 15B are isometric views showing an elongated element pushing forward in a distal direction to bend a distal portion of the shaft, in accordance with one embodiment of the present invention;

FIG. 15AA is an isometric view similar to FIG. 15A except that instead of cut-outs 33 there are closed slits 55, in accordance with one embodiment of the present invention;

FIG. 16A and FIG. 16B are isometric views showing a rigid elongated element straightening out a non-straight longitudinal channel, in accordance with one embodiment of the present invention;

FIG. 17 is a flow chart of a further method, in accordance with one embodiment of the present invention;

FIG. 18 shows a screw used in sacroiliac joint fixation, in accordance with one embodiment of the present invention;

FIG. 19 shows a screw used in fixation for ALIF implants, in accordance with one embodiment of the present invention; and

FIG. 20 shows a screw used in femoral neck fixation, in accordance with one embodiment of the present invention.

### DETAILED DESCRIPTION OF THE INVENTION

The following detailed description is of the best currently contemplated modes of carrying out the invention. The description is not to be taken in a limiting sense, but is made merely for the purpose of illustrating the general principles of the invention, since the scope of the invention is best defined by the appended claims.

The present invention generally provides a method and apparatus for an anchoring element, such as a bone screw, with a deflectable portion to increase the pullout resistance of the anchoring element and/or its resistance to loosening. The anchoring element may initially assume a straight configuration in which it behaves as a conventional anchoring element, for example a screw being inserted by a normal threaded insertion method in which the helical thread engages the bone tissue (if it is a bone screw) and the screw advances. The shaft may have threading along at least a portion of its length and may have a core with at least one longitudinal channel for housing an elongated element that may be a tensioning element or a rigid reinforcing element or both. The longitudinal channel may be displaced off center in at least one dimension (vertically or horizontally) along a plane perpendicular to the longitudinal axis of the shaft. The elongated element may traverse at least one of the one or more lateral cut-outs (or at least one side of one of the cut-outs) in the core of the shaft, which may be located at the distal portion of the shaft, or in some embodiments at the central portion of the shaft. The cut-outs may be backed by an effective hinge, which may be an integral hinge. The elongated element and the distal portion may be configured such that upon axial movement of the elongated element through the channel toward the proximal end of the shaft (or toward the distal tip of the shaft in other embodiments), the distal portion may deflect to an angle from the longitudinal axis. If the elongated element is a tensioning element it may be affixed to the distal portion. If the anchoring element is pre-biased to deflect, the elongated element may be rigid non-tensioning element that when moved axially allows the anchoring element to leave its straightened configuration and deflect. In another version, the elongated element may be pushed through a non-straight or curved or contoured longitudinal channel to straighten the channel and thereby effectuate the deflection of the distal portion of the shaft. After the anchoring element has been fully inserted, upon axial movement of the elongated element, a bend or other deflected shape may be formed, preferably at or near the distal portion, such as a distal tip, of the anchoring element. Non-limiting medical applications include a bone screw used in sacro-iliac joint fixation, fixation for ALIF implants, lateral approach implants, femoral neck fixation and percutaneous humeral head fixation. A non-limiting example of a valuable non-medical mechanical application includes passing a screw through a sheet of material, such as a hollow wall structure, and then deflecting the distal portion in order to improve anchoring of the screw.

In contrast to prior art anchoring elements such as bone screws, in which the pullout strength of a screw and its resistance to loosening are limited by the fact that the shaft of the screw is straight, the present invention may have an increased pullout strength and resistance to loosening. The deflected part of the anchoring element may greatly increase the effective area resisting axial withdrawal (“pullout”) of the anchoring element. In addition, the risk of undesirable loosening or failure of the anchoring element may be reduced due to the decreased cyclic and/or static loading on the deflected part. The “loosening” that the present invention may inhibit is one or both of the following: (a) reduced fixation to the tissue (i.e. the anchor or screw can wiggle) and (b) the anchor, i.e. screw, unthreads from the tissue by rotating along the long axis to the point where it ‘backs out’ of the tissue. In further contrast to the prior art bone anchoring elements, such as bone screws, which may have a straight shaft, the anchoring element of the present invention may have a distal portion, such as a distal tip, that is bent in relation to a longitudinal axis of the shaft of the anchoring element. In further contrast to methods of the prior art, in which anchoring elements such as bone screws are inserted into tissue, and their shape is not altered, a method of the present invention, may in some preferred embodiments include deflecting a distal portion, such as a distal tip, of the bone screw after it has been inserted. In some preferred embodiments, the method the present invention may include straightening out the bent distal portion of the anchoring element prior to removal of the anchoring element. In still further contrast to prior art anchoring elements such as bone screws, in which the core of the shaft may be solid, the core of the shaft of the anchoring element of the present invention may have cut-out portions, especially lateral cut-out portions. The cut-out portions may be backed by an effective hinge that may be an integral hinge. The effective hinge may be displaced from the longitudinal axis to improve leverage. The cut-out portions may have associated transverse slits to facilitate deflection. These cut-out portions may be V-shaped, U-shaped or of another shape in cross-section and may be more proximal than the distal tip. In still further contrast to certain prior art anchoring elements, in which slots may be formed longitudinally in the anchoring element, the anchoring element of the present invention, may, in one preferred embodiment, have cut-outs that may be lateral cut-outs in the core of the anchoring element. In yet still further contrast to the prior art, in which an expansion of a portion of an anchoring element may be such that the expansion is symmetrical and/or may involve a plurality of segments expanding outward symmetrically, the anchoring element and method of the present invention, in one preferred embodiment, may involve a deflectable portion or deflectable distal end, or a deflectable distal tip, that may comprise an asymmetric deflection, for example in that an end view of the anchoring element in its already deflected configuration may be asymmetric. In further contrast to the prior art, in which an expansion of an anchoring element may involve a plurality of separated portions of the anchoring element connected to a common base and expanding identically and simultaneously, the anchoring element and method of the present invention may, in one preferred embodiment, involve successive deflection of segments of one distal portion of the anchoring element such that a particular segment of the deflected portion, taken alone, may be positioned at a different deflection angle to the longitudinal axis than that of another segment of the deflected portion. In further contrast to the prior art, in which the shaft of the anchoring element is solid, the shaft of the bone screw of the present invention may have one or more longitudinal channels. The channel(s) may be on a surface of the shaft or may be entirely within the core of the shaft. The channel may be off center from a longitudinal axis of the shaft. In still further contrast with the prior art anchoring elements such as bone screws, the method and anchoring element of the present invention may utilize an elongated element, that may be a tensioning element, affixed to a distal portion, such as a distal tip, of the anchoring element. The elongated tensioning element may be pulled or pushed to deflect the distal portion of the anchoring element at an angle that in some preferred embodiments is between 10 and 180 degrees (or in other preferred embodiments between about 70 and about 110 degrees) from the longitudinal axis of the shaft. In contrast to prior art methods, in which the bone screw displaces trabecular bone and the displaced spongy bone forms a thickened bony envelope around the shaft of the bone screw, the method of the present invention may, after deflection of the distal tip, yield an area adjacent the distal tip that is void or of reduced or very low bone density on one side of the distal tip. This may reduce cyclic loading on the deflected part and reduce the risk of the screw loosening and/or failing. In contrast to the prior art screws and methods, the deflected tip may also yield an area of increased concentration adjacent the deflected distal tip on an opposite side of the distal tip. In still further contrast to the prior art, a method of the present invention may, in some preferred embodiments, include resiliently pre-biasing the anchoring element to assume the deflected form and then using a rigid reinforcing element to hold the anchoring element straight until removal of the rigid reinforcing element. This may be useful where additional rigidity is required during threaded insertion of a screw into a bone. In further contrast to the prior art, in some preferred embodiments, the anchor or screw, or in other preferred embodiments, the hinges alone, may be made of a material that is a shape memory alloy (i.e. Nitinol or another such alloy) such that the deflectable portion is provided in a default deflected state. In that case, a rigid element positioned in the core of the screw may hold the deflectable portion in a straight configuration for insertion into the bone. After positioning, the rigid element may be removed and the deflectable portion (with or without the aid of an elongated element (tensioning or rigid reinforcing) being pulled) may deflect to its bent configuration. In still further contrast to prior art anchoring elements, in which a channel may be static, the present invention, in some preferred embodiments, utilizes a longitudinal channel that before axial movement of the elongated element may be in a contoured or non-straight configuration whereas the channel may be straight after axial movement of the elongated element.

The principles and operation of an apparatus and method for a bone screw with a deflectable portion according to the present invention may be better understood with reference to the drawings and the accompanying description.

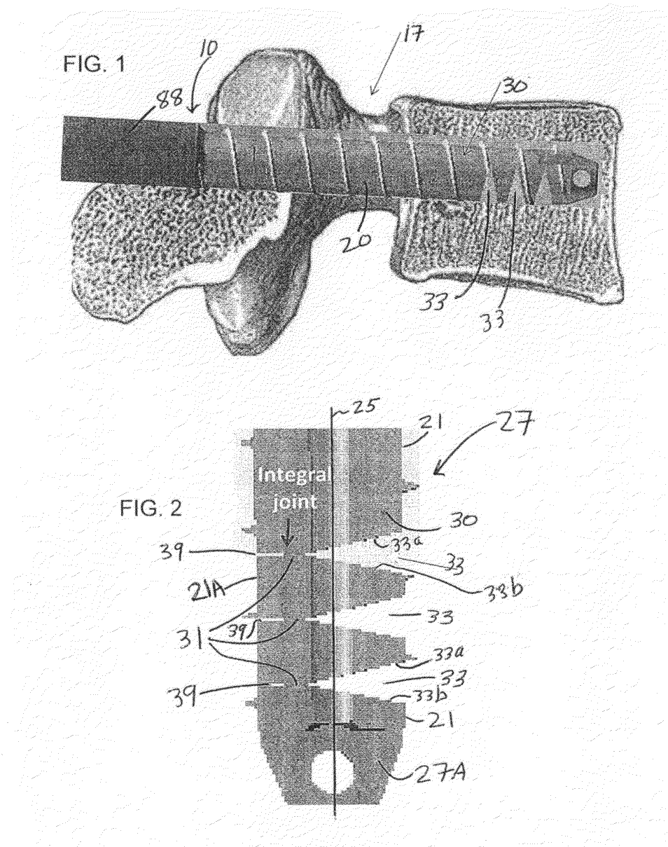

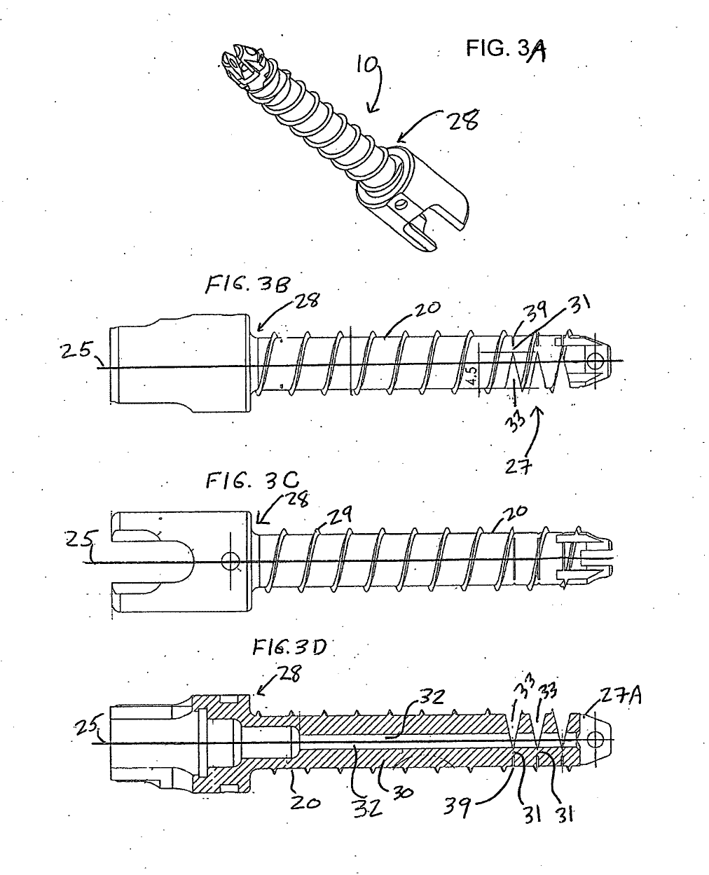

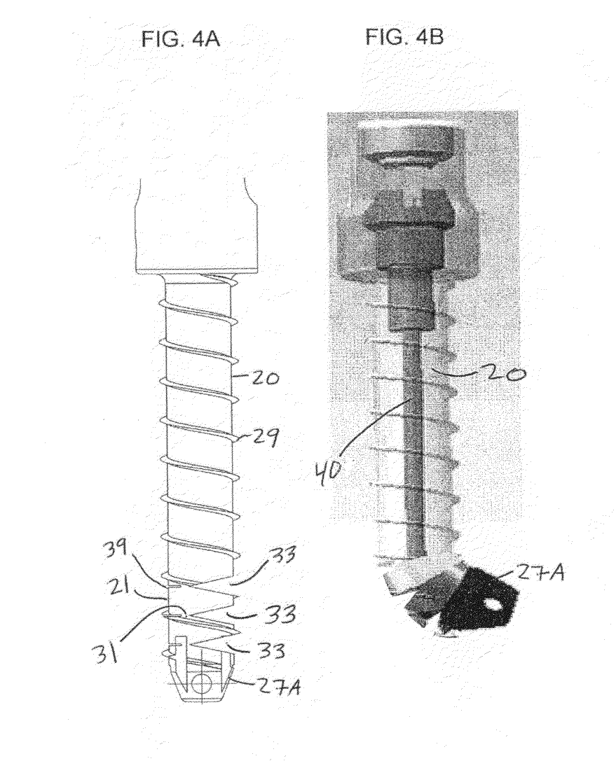

As shown in FIG. 1 through FIG. 3D, an anchoring element 10, such as a bone screw 10, may comprise a shaft 20 having a core 30 that may be cannulated to define one or more longitudinal channels 32 (see FIG. 3D) (also called a lumen). The longitudinal channel or channels 32 need not be situated at the center of shaft 20 or of core 30 but may be situated at a distance from (i.e. displaced from) the longitudinal axis 25 (see FIG. 3C) (which longitudinal axis 25 is at the center of the shaft 20 or anchoring element 10). This displacement may be vertical and/or horizontal along a plane (not shown) perpendicular to the longitudinal axis 25 of shaft 20. In other preferred embodiments, the channel 32 is on the longitudinal axis 25 in the center of the shaft 20. As shown in FIG. 2, shaft 20 may have a distal portion 27 (which for example may be the last 10%, the last 20%, the last 30%, the last 40%, the last 50% or some other last portion of the screw or anchoring element 10, in various different preferred embodiments) that may be subject to deflection. Shaft 20 may have an outer surface 21.

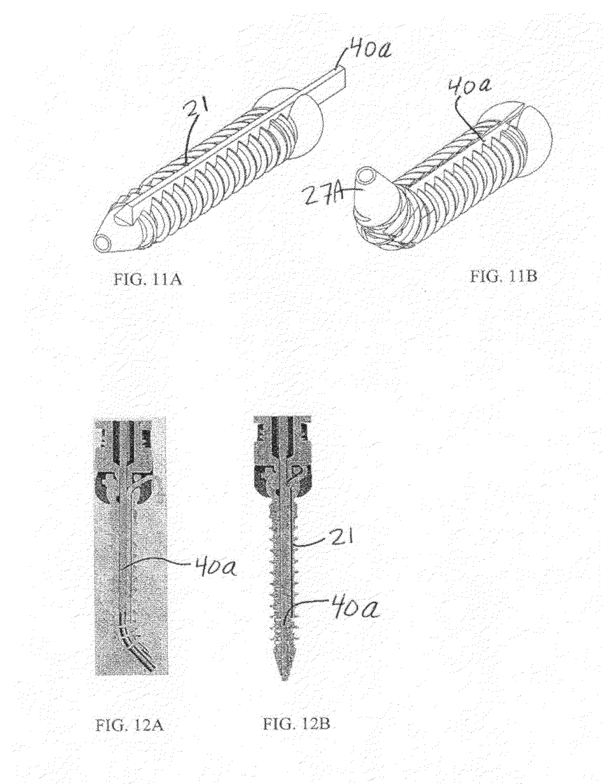

Longitudinal channel 32 may also be on the surface 21 of shaft 20. Although an example of this is shown in FIGS. 11A and 118 and FIGS. 12A and 12B, which is a case where the elongated element 40a is not a tensioning element, this should not be understood as any limitation and channel 32 may be on the surface of shaft 20 when elongated element is a tensioning element or in any other suitable embodiment.

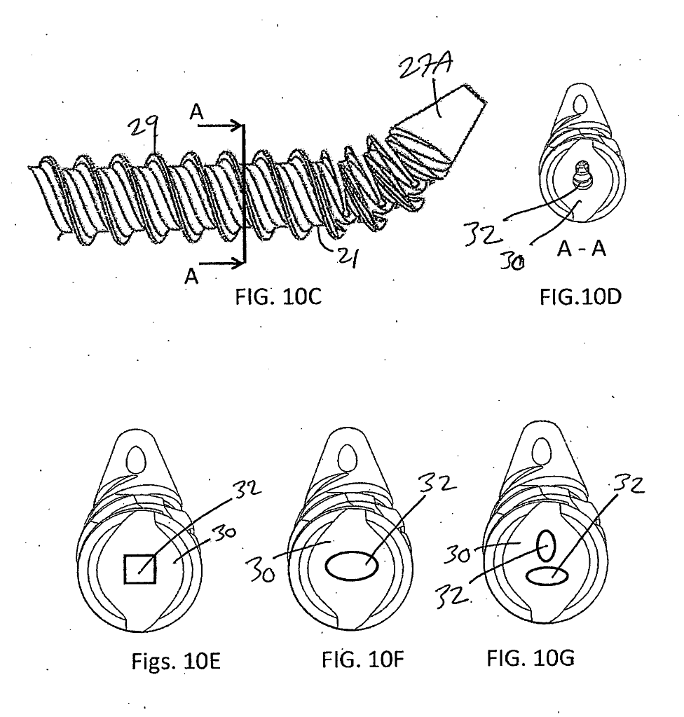

FIG. 10D and FIG. 10G show cases of shaft 20 having multiple longitudinal channels 32. FIGS. 10C through 10G also show examples of longitudinal channels 32 whose cross-section is not round, but rather for example square (FIG. 10E) and elliptical (FIG. 10F and FIG. 10G).

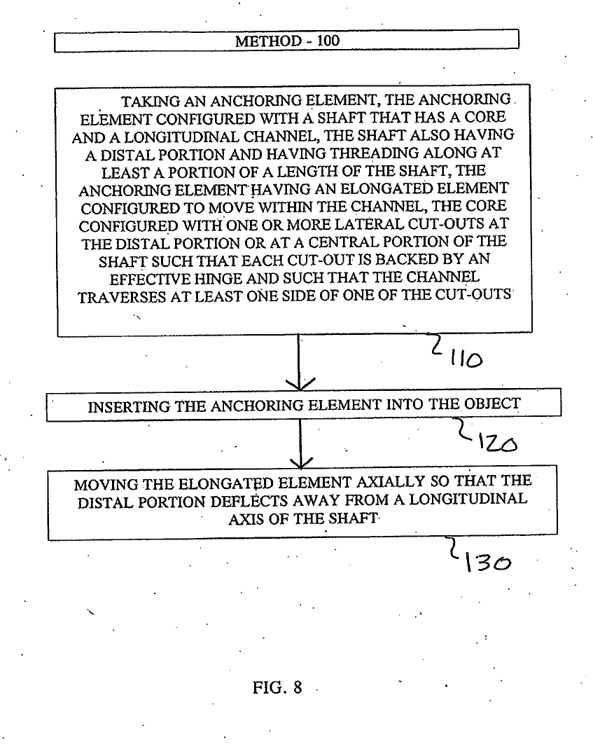

As shown in FIG. 2, the core 30 may have one or more cut-outs 33 at the distal portion of the shaft (which may also be the distal end of core 30). As seen schematically from FIG. 7C, it is also possible that core 30 may have one or more of the cutouts 33 at a central portion 35 of shaft 20, particularly where there is only one cut-out 33 and the distal portion 27 is roughly half the length of the shaft 20. In FIG. 7C, the single cut-out 33 is in a central portion 35 of shaft 20 but is also partly in a distal portion 27 of shaft 20 since the distal side 33b of cut-out 33 is in the portion that deflects. Accordingly, in accordance with the present invention, one or more cut-outs 33 may be at the distal portion 27 or central portion 35 of shaft 20, which is understood to refer to at the distal portion 27 or at the central portion 35 or at both, since it is also possible that a cut-out 33 can be partially in the distal portion 27 and partially in the central portion 35 of shaft 20. As shown in FIG. 2, the cut-outs 33 may be V-shaped in cross-section. The number of cut-outs 33 may vary. As FIG. 2 shows, in one preferred embodiment, cut-outs 33 may be lateral cut-outs into core 30 along surface 21 of shaft 20. In a different preferred embodiment, core 30 may have one or more cut-outs 33 at a distal portion of a threaded portion of the shaft 20.

Lateral cut-outs 33, in one preferred embodiment, span 60 rotational degrees or less between an upper part 33a of a cut-out and a lower part 33b of the cut-out (as measured from a point of rotation at the deepest point of the cut-out 33). In other preferred embodiments, the rotational degrees spanned is 70 degrees or less or in other preferred embodiments 80 degrees or less or in other preferred embodiments 90 degrees or less. These amounts of rotational degrees are merely illustrative non-limiting examples.

Cut-outs 33 may be close to a distal portion, for example a distal tip 27a, of the shaft 20 (which is also the distal portion 27, for example the distal tip 27a, of the anchoring element 10 in general) and may be proximal in relation to the distal portion 27 of shaft 20 that may be subject to deflection. Cut-outs 33 may be symmetrical about a plane (not shown) that is at 90° to the longitudinal axis (or in some other preferred embodiments substantially symmetrical, i.e. plus or minus 15% from the plane, or in other preferred embodiments not symmetrical about such a plane). Cut-outs 33 may interrupt the helical thread 29 around the shaft 20, assuming the anchoring element 10 is a screw. Even if the cut-outs 33 form interruptions in the helical thread 29, the cut-outs 33 do not significantly impact function of the helical thread as a whole during insertion of the screw 10.

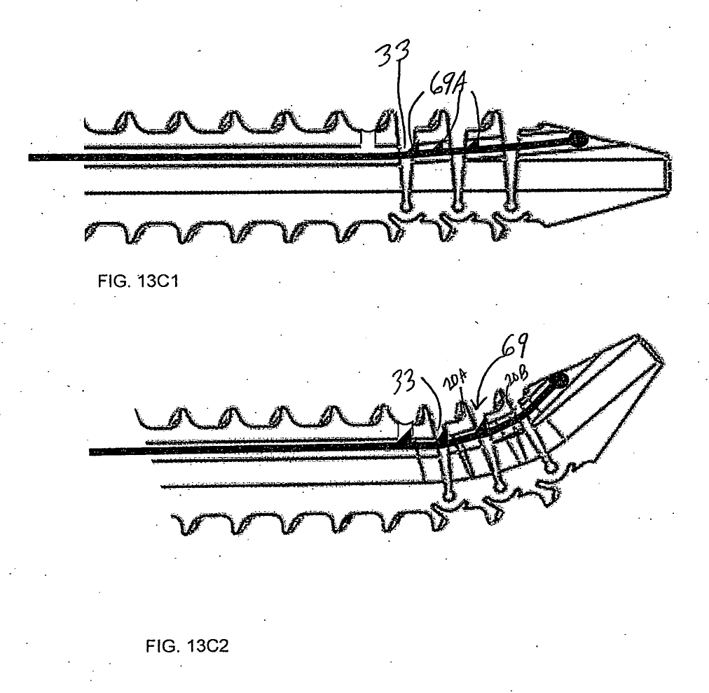

It should be understood that the present invention does not place limitations on the location or nature of any threading or other resisting surface that may appear on the anchoring element 10 or on its surface 21 (or whether there will necessarily be any threading). For example, any such threading 29 may be on the entire length or along one or more portions of the lengths of the shaft or excluding particular portions thereof (for example on the entire shaft other than the distal tip 27 or other than the distal end 22 or other than the deflectable portion). The threading 29, if any, may also have a variety of dimensions (in terms of pitch, tooth shape, tooth size or other dimensional features), may include a double thread, and further may have various (i.e. left or right) coil directions. FIGS. 13a-13B show a preferred embodiment in which there is threading along at least a portion of the length of the shaft 20.

Each cut-out 33 may be backed by an effective hinge 31 comprising a connection arrangement between cut-outs 33 allowing a pivoting movement between one side of a cut-out (i.e. 33a) and the other side of that cut-out (i.e. 33b) in the substantially axial direction. The movement may pivot on a part of effective hinge 31 that has little longitudinal dimension as shown in FIG. 2, but in other examples of effective hinge 31 the movement may pivot on a strip of material of effective hinge 31 having a more substantial longitudinal length. Although FIG. 2 shows that each cut-out 33 (also called a wedge 33) may be backed by an effective hinge that is an integral hinge 31, a non-integral hinge is also contemplated. In order to facilitate the deflection and provide leverage to the elongated tensioning element 40, each effective hinge 31 may be displaced from the longitudinal axis 25. FIG. 2 further shows that there may be a transverse relief slit 39 (or fissure 39) between one or more effective hinges 31 (or all of the effective hinges 31) and a surface 21A of shaft 20 that may be opposite a cut-out 33 backed by the respective one or more effective hinges 31, that may be integral hinges 31. The cut-out wedges 33 may also be shaped such that a curved effective hinge 31A is made, as shown in FIG. 10A and FIG. 10B. As shown by FIGS. 10A-10B, the cut-outs 33 may result in shaft 20 to be divided essentially into segments 20A, 20B, 20C, etc., within distal portion 27, for example distal tip 27a, of shaft 20. In FIG. 10A, three segments have been designated as 20A, 20B, 20C. In the embodiment shown in FIGS. 10A and 10B, no relief slits 39 are shown since effective hinge 31A is at the surface 21 of shaft 20.

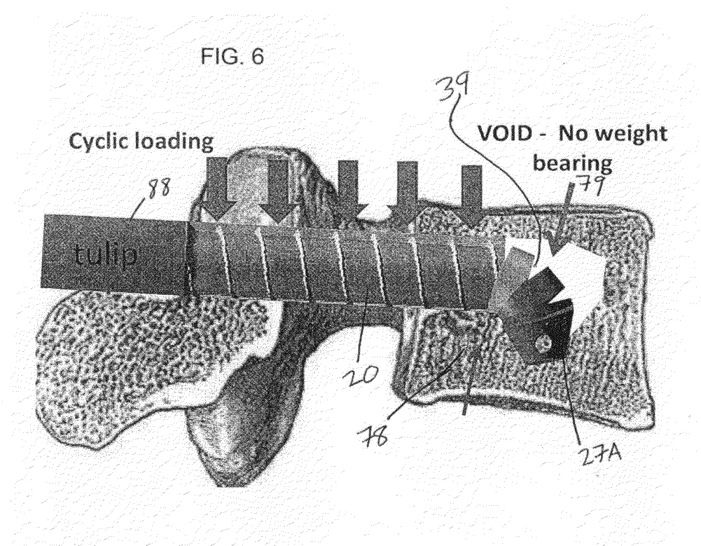

Each effective hinge 31 may have the associated relief slit 39. Accordingly, the effective hinge 31 may be the material between the slit 39 and the cut-out 33. These relief slits 39 may increase the flexibility of the shaft 20 structure in the direction of deflection. During deflection, as shown in FIG. 5, these relief slits 39 may open up as the wedge-shaped cut-outs close. FIG. 5 shows the closed wedges 33 or cut-outs 33.

The longitudinal channel 32 in core 30 (see FIG. 3D) of anchoring element 10 may house an elongated element 40 that may move axially within channel 32. The elongated element 40 and the distal portion 27 may be configured such that upon axial movement of the elongated element (in one preferred embodiment toward the proximal end 28 of shaft 20 or in another preferred embodiment toward a distal tip 27A of the shaft 20), the distal portion 27 of the shaft 20 may deflect to an angle from the longitudinal axis of the shaft. In a preferred embodiment, elongated element 40 may traverse at least one of the cut-outs 33, or at least one side (i.e. 33a or 33b) of one of the cut-outs 33. In one preferred embodiment, elongated element 40 traverses more than one of the cut-outs 33.

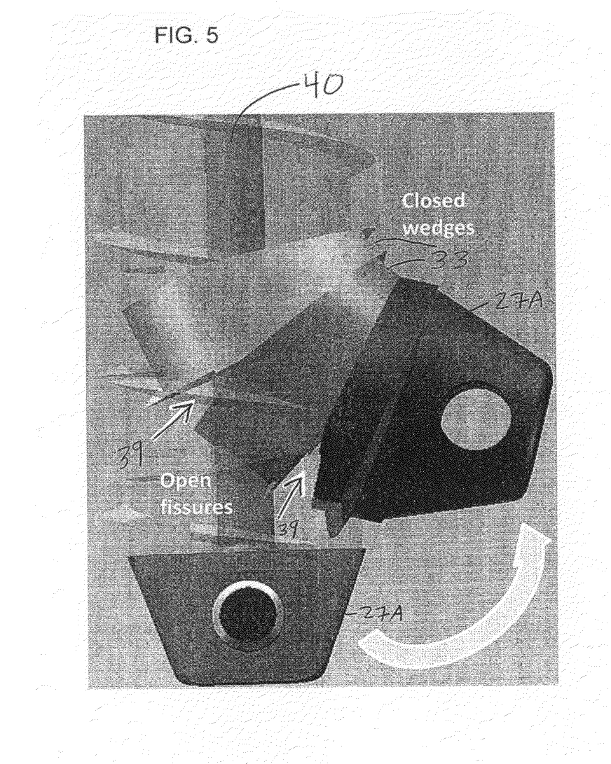

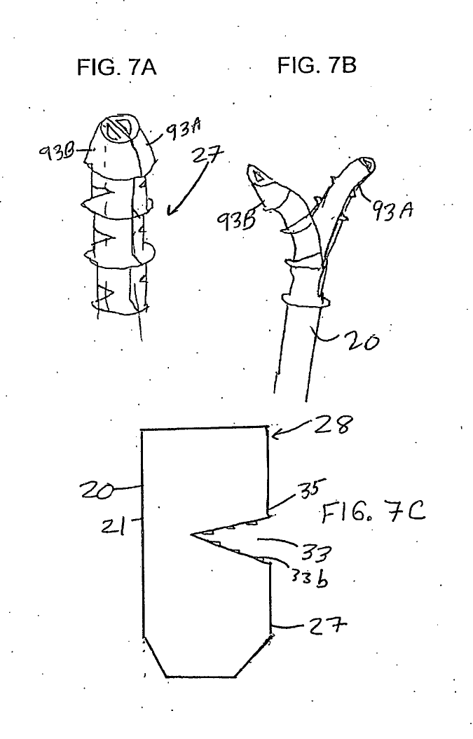

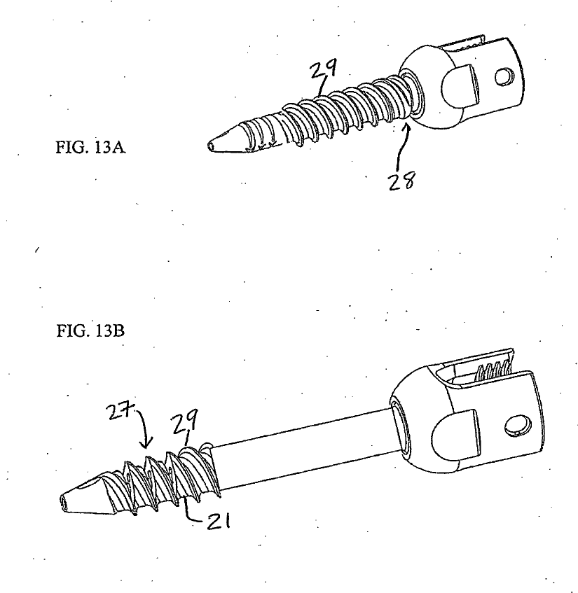

The elongated element 40 may be a tensioning element 40 (best seen in FIGS. 10A and 10B) and may be affixed to the distal portion 27, such as a distal tip 27a of the shaft 20, which may also be a distal portion 27 or tip 27a of anchoring element 10. The tensioning element 40 can be a non-rigid drawstring or may be a rigid elongated element with sufficient rigidity to apply tension/or compression, as described herein. If the elongated tensioning element 40 is located in longitudinal channel/lumen 32 that is off center, that may allow a further central channel that is on the longitudinal axis 25 to be used for other purposes, for example as a passageway for a k-wire. Due to the cut-outs 33 and effective hinges 31 and, and in some preferred embodiments due also to the transverse relief slits 39, the distal portion 27, for example the distal tip 27A, of the shaft 20 may be configured, upon tension from the elongated tensioning element 40, to deflect to an angle from the longitudinal axis of at least 10 degrees. For example, as shown in FIG. 4B, FIG. 5 and FIG. 6, elongated tensioning element 40 may be pulled axially in a direction proximal to the distal portion 27, for example distal tip 27a, to cause the tension that deflects distal portion 27. Deflection of the distal portion 27, for example distal tip 27a may be activated when the pull of the elongated tensioning element 40 closes shut (or nearly shut) the cut-outs 33 (wedge spaces) and opens wide the slits 39 (fissures). The distal portion 27 may be re-straightened by pushing elongated tensioning element 40 axially in a direction toward distal portion 27, i.e. in a direction opposite to the direction that tensioning element 40 was originally moved/pulled in to cause the deflection. Alternatively, distal portion 27 may be re-straightened by a different tensioning element, which may be situated in a different longitudinal channel, may be pushed axially in the direction toward distal portion 27, i.e. opposite to the direction that tensioning element 40 was originally moved in to cause the deflection. In a further alternative, distal portion 27 may be re-straightened by pulling a different tensioning element located on the side of the shaft axis 25 opposite to the deflection. In a still further alternative, anchoring element 10 may also be straightened by axially moving a rigid reinforcing element (such as the rigid element 40a shown in FIG. 12A-12B) that is not a tensioning element that may be situated in a separate channel, which may be a central channel. Furthermore, even if elongated element 40 is a tensioning element, in certain preferred embodiments elongated element 40 need not be affixed to the deflecting distal portion 27.

As shown in FIG. 11A and FIG. 11B, the elongated element 40a may be a rigid elongated reinforcing element 40a that is not a tensioning element. In this case, the rigid elongated reinforcing element 40a may be situated in a longitudinal channel 32 on a surface 21 or perimeter of shaft 20 or of anchoring element 10 and may be an independent element that may not be affixed to the deflected distal portion 27, such as distal tip 27A. As shown in FIG. 12A and FIG. 12B, the rigid elongated reinforcing element 40a may be in a channel 32 that is a central channel in that it is situated centrally with respect to one dimension of a plane (not shown) perpendicular to the longitudinal axis 25 (see FIG. 3C). In other preferred embodiments, the element 40a may be in a channel entirely inside the core 30 of shaft 20. The method 200 below describes steps for using a rigid reinforcing element 40a for deflection of the distal portion 27 of the anchoring element 10. The cross-section of rigid elongated reinforcing element 40a and corresponding elongated longitudinal channel 32 may be of any suitable shape, such as dovetail, round, etc.

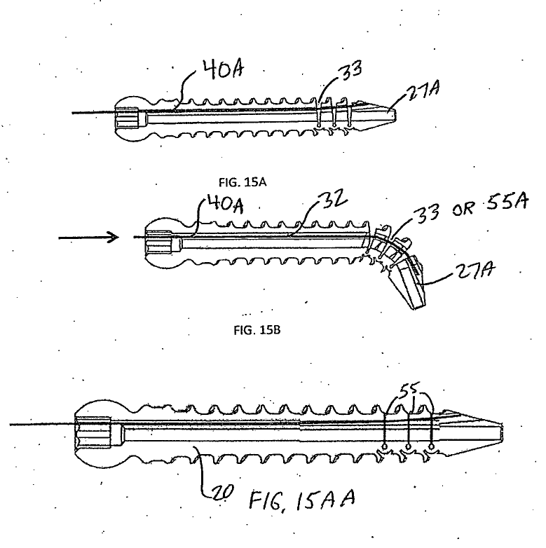

As shown in FIG. 15A and FIG. 15B, an elongated reinforcing element 40a may be moved axially within longitudinal channel 32 (for example by pushing in a distal direction as shown in the arrow in FIG. 15B) to deflect distal portion 27, for example a distal tip 27A. In this case, the deflection is to the side of the shaft 20 opposite to that of the cut-outs 33.

It should be understood that the terra lateral “cut-outs” 33 as used in this patent application does include lateral slits or other lateral openings even if no material has been removed from the core 30 to create the slit or opening and also includes lateral cut-outs where material has been removed. In some preferred embodiments of the version discussed in FIGS. 15A-B, there may be closed lateral slits 55 (see FIG. 15AA) in a distal portion or in a central portion of the shaft 20 in the straight configuration (similar to that shown in FIG. 15A) and it may be that no material of the core 30 is actually “removed”. In the deflected configuration of FIG. 15B, the closed slits 55 of FIG. 15AA open. Note that although elongated reinforcing element 40a has enough rigidity to effectuate the deflection shown in FIG. 15B, this elongated element 40a is sufficiently flexible to bend during deflection and hence may be described as semi-rigid or somewhat rigid or having a sufficient rigidity to bend but to also effectuate the deflection. Since the configuration of FIG. 15B may be reached from an initial configuration shown in FIG. 15A (cut-outs 33 that open further) or from the initial configuration shown in FIG. 15AA (closed slits 55 becoming open slits 55A that are cut-outs 33), the “cut-outs” in FIG. 15B have been labeled “33 or 55A”. Elongated reinforcing element 40a may be unattached to distal portion 27, for example distal tip 27A, although this is not a limitation.

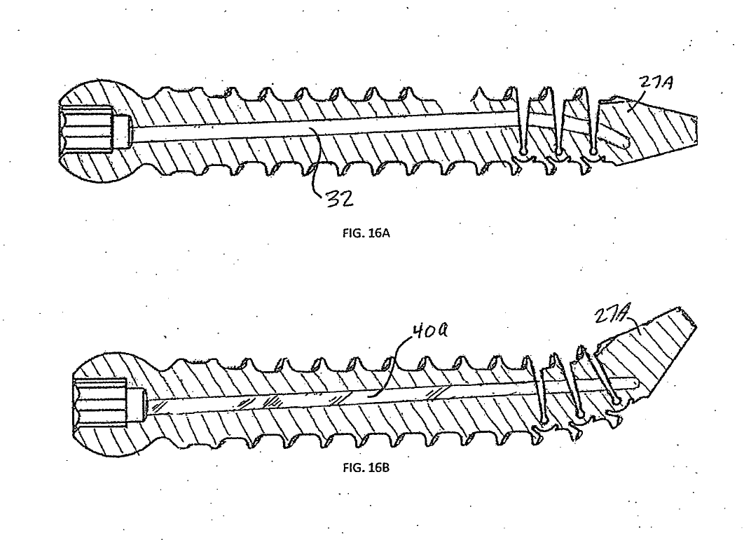

As shown in FIG. 16A and FIG. 16B, in a further preferred embodiment for deflection of distal portion 27, for example distal tip 27A, typically used for deflection within a limited angle, a longitudinal channel 32 may be contoured or biased (see FIG. 16A) and moving a rigid elongated element 40a axially (for example by pushing in a distal direction) within the contoured longitudinal channel 32, for example in a central channel, may straighten out channel 32 and thereby cause deflection of distal portion 27. The elongated element 40a and channel 32 may be configured such that pushing the elongated element 40a through the longitudinal channel 32 toward a distal tip 27a may deflect the distal portion 27, for example distal tip 27A, by putting the longitudinal channel into a straight configuration from a non-straight configuration

Deflection of the distal portion 27 of the anchoring element 10 may be performed in any direction. According to one particularly preferred option where the anchoring element is a bone screw, it may in some cases be advantageous to employ deflection downwards (in a caudal direction), since this may minimize the chance of normal axial loading of the screw 10 resulting in loading of the deflected tip portion. The deflection shown in FIGS. 10B, 11B, 12B may be described as asymmetrical in that an end view of the deflection would show the deflected portion 27 bending away from the longitudinal axis asymmetrically, for example in one direction.

The angle of deflection from the longitudinal axis 25 may be between about 10 degrees (or less) and about 180 degrees or more. In some preferred embodiments, this angle is between about 15 and about 150 degrees, or in other preferred embodiments between about 50 and about 130 degrees or on still other preferred embodiments between about 70 degrees and about 110 degrees, or other combinations of these numbers (i.e. about 50 degrees and about 110 degrees). Although the drawings show a fully deflected configuration that turns through roughly 90 degrees, i.e., between about 70 degrees and about 110 degrees, alternative embodiments in which the deflected portion turns through smaller angles (e.g., 30-70 degrees) or larger angles (e.g., 110-180 degrees) also fall within the broad scope of the present invention. Depending upon the type of tissue involved and the geometry of deployment relative to the tissue, even smaller deflection angles, such as a 10 degree deflection, may be effective to achieve enhanced resistance to pullout and/or loosening of the device.

In certain preferred embodiments, as seen from FIG. 13C1 and FIG. 13C2, anchoring element 10 may further comprise a locking mechanism 69 for maintaining deflection of the distal portion 27, such as distal tip 27A. For example, elongated tensioning element 40 or elongated reinforcing element 40a may have a series of projections, such as inclined teeth 69A, that may correspond to and may get caught or stuck in cut-outs 33 in core 30 for locking engagement. This would prevent the segments (for example segments 20A, 20B) from returning to their initial state before deflection. In another version of locking mechanism 69, a projection (or recess) on channel 32 may catch a groove or recess (or projection) on elongated element 40 or 40a and prevent the elongated element (40 or 40a) from moving further once deflection of distal portion 27 occurs.

In a case where the anchoring element 10 is a screw, the screw typically has a “tulip” (tulip-shaped hollow connector 88 (see FIG. 1, FIG. 6)) on the rear or proximal end of the shaft to accommodate a rod that may connect two or more pedicle screws. The tulip 88 may be monoaxial or it may swivel (polyaxial) or be monoplanar. The tulip 88 may house an elongated tensioning element 40 or drawstring which extends down to the distal portion 27, such as distal tip 27a—and a mechanism (typically a threaded tightening engagement between an element connected to the elongated tensioning element 40 and a rotatable nut) to tighten it. Tightening elongated tensioning element 40 makes the distal segmented portion of the screw change its shape into a curved/deflected configuration.

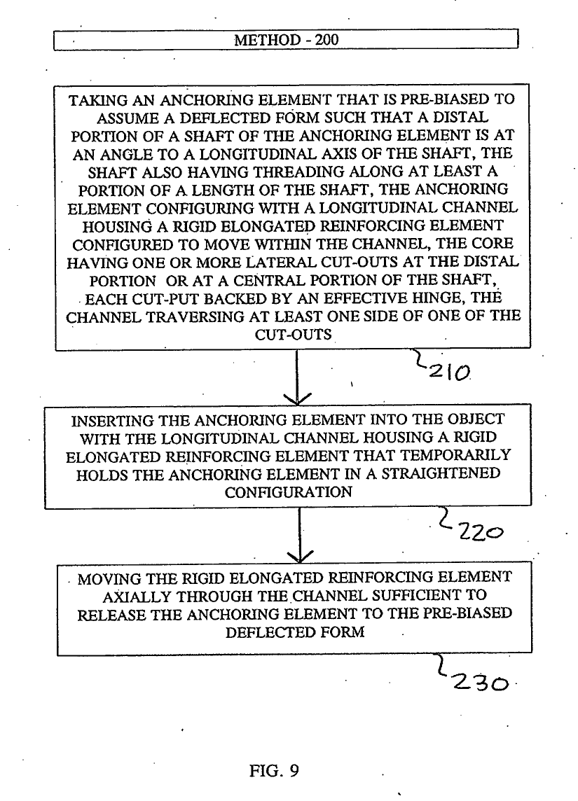

As shown in FIG. 8, the present invention may also be described as a method 100 of inserting an anchoring element into an object. Method 100 may comprise a step 110 of employing or taking an anchoring element, the anchoring element configured with a shaft that has a core and a longitudinal channel (or more than one longitudinal channels), the shaft also having a distal portion and having threading along at least a portion of a length of the shaft, the anchoring element having an elongated element (which may be a tensioning element or may be a rigid element) configured to move within the channel, the core configured with one or more lateral cut-outs at the distal portion 27 or at the central portion 35 of the shaft 20 (which is understood to include a case where a cut-out 33 is in both portions 27, 35) such that each cut-out is backed by an effective hinge (which may be an integral hinge) and such that the channel 32 traverses at least one of the cut-outs 33, or at least one side (i.e. 33a or 33b) of one of the cut-outs. The longitudinal channel (or more than one such channel) may be substantially parallel to a longitudinal axis of the shaft 20. In some preferred embodiments, the core may include a further step of configuring the core with a transverse slit on a surface of the core opposite a cut-out, for one or more of the cut-outs, to facilitate deflection of the anchoring element. Method 100 may also have a step 120 of inserting the anchoring element into the object. Method 100 may also include a step 130 of moving the elongated element axially toward a proximal end of the shaft so that the distal portion 27 deflects away from the longitudinal axis. It should be understood that “axial movement” refers to movement in the longitudinal direction of the anchoring element 10. In a further preferred embodiment, method 100 may include a step of moving the elongated element axially toward a distal end of the shaft so that the distal portion 27 deflects away from the longitudinal axis.

Longitudinal channel 32 may be considered longitudinal notwithstanding the fact that particular segments of channel 32 are not strictly longitudinal if the main overall direction of the channel is longitudinal. This may be particularly applicable to the case where channel 32 houses a non-rigid tensioning element 40, such as a drawstring. For example, a channel extending along a length of shaft 20 and configured as a wave or a flattened “W” or along a winding path would still be longitudinal since the main overall direction of the channel is longitudinal. In one preferred embodiment, the anchoring element 10 or method (100, 200) of the present invention may employ a channel 32 where the effective force of a tensioning element 40 such as a drawstring through the channel 32 is to exert a substantially longitudinal force on the deflecting distal portion 27 even though the shape of the channel 32 is not strictly longitudinal or not longitudinal.

The deflecting of the distal portion 27, such as distal tip 27A, may be so that the distal portion 27 is at an angle from the longitudinal axis of between about 10 degrees and about 180 degrees, or at any other angle described herein, such as between about 70 and about 110 degrees. Some versions of method 100 may also include a step of deflecting the distal portion 27 until the cut-outs close together. Some versions of method 100 include a step of straightening the deflected distal portion 27 by pushing the elongated element (40 or 40a) axially in a distal direction until the anchoring element returns to an original straight configuration. Alternatively, the straightening of the deflected distal portion 27 may be achieved by pushing a rigid rod into the cannula or longitudinal channel in the shaft and pushing the segments back into a straight configuration. Then, an expanded version of a method of inserting into and removing from an object and anchoring element may include a step of removing the anchoring element from the object.

As shown in FIG. 16A and FIG. 16B, in some preferred embodiments, moving the elongated element axially so that the distal portion deflects away from a longitudinal axis of the shaft may be effectuated by pushing the elongated element through a longitudinal channel to put the longitudinal channel into a straight configuration from a non-straight configuration. For example, as shown in FIG. 16A, channel 32 may be contoured, curved or biased prior to insertion of the elongated element 40a and may be straight after insertion of the elongated element 40a. Elongated element 40a may be rigid to effectuate this straightening of channel 32.

It is particularly significant to note in regard to an anchoring element that is a bone screw that, as shown in FIG. 6, the deflected portion of the screw generally creates, and is therefore adjacent to, a void 79 (or at least, a region of very low bone density 79), so that the deflected portion typically does not take a major role in bearing axial or other forces resulting from normal static and/or cyclic loading of the bone screw as it performs its primary intended load-bearing function. On the other hand, the region of bone proximal to the final deflected position of the tip has been to some degree compressed during the deflection motion, leading to a locally increased trabecular concentration which enhances resistance to pull-out forces. Accordingly, in some versions of method 100, there is a further step of reducing axial loading on the distal portion 27, such as on distal tip 27A, by having the deflecting create a region of very low bone density in the object adjacent the deflected distal portion 27, such as distal tip 27A. Another step of method 100 may be creating an area 78 of increased concentration in the object adjacent the deflected distal portion or tip on an opposite side of the distal portion or tip from the region of very low bone density.

In some preferred embodiments described in this document, the screw can be re-straightened for withdrawal if clinically necessary. For example, in some cases, reversing the tightening mechanism within the tulip preferably forcibly returns the screw to its original straight configuration, allowing removal of the screw in a conventional manner if required. Where a flexible drawstring is used, reversibility of the tip deflection may be ensured through the elastic property of the resilient material of the screw. More preferably, the “drawstring” is implemented as a semi-rigid rod which is effective to actively deflect the bent tip back to its straightened state.

It should be noted that the deflection of the screw may be performed elastically, or part or all of the motion may include plastic deformation. In the latter case, an additional step of plastic deformation will typically be required if the screw is to be returned to its straightened configuration for removal.

Optionally, where additional rigidity is required during threaded insertion of the screw into the bone, the screw may be formed with an inner passageway which temporarily houses a rigid reinforcing element such as a K-wire (not shown) during insertion of the screw. According to this option, the screw may optionally be resiliently pre-biased to assume its deflected form, and may be temporarily retained in its straightened configuration by the presence of the reinforcing element until after insertion.

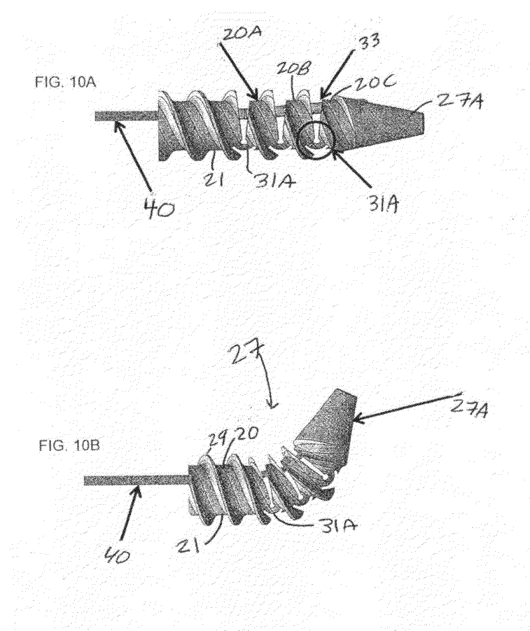

Accordingly, as shown by FIG. 9, the present invention may also be described as a further method 200 of inserting an anchoring element into an object. Method 200 may comprise a step 210 of taking or employing an anchoring element that is pre-biased to assume a deflected form such that a distal portion of a shaft of the anchoring element is at an angle to a longitudinal axis of the shaft, the shaft also having threading along at least a portion of a length of the shaft, the anchoring element configuring with a longitudinal channel housing a rigid elongated reinforcing element configured to move within the channel, the core having one or more lateral cut-outs at the distal portion or at a central portion of the shaft (which is understood to include a case where a cut-out 33 is in both portions), each cut-put backed by an effective hinge (which may be an integral hinge), the channel traversing at least one of the cut-outs, or at least one of the sides (i.e. 33a or 33b) of one of the cut-outs 33.

Step 220 of method 200 may involve inserting the anchoring element into the object with the longitudinal channel housing a rigid elongated reinforcing element 40a that temporarily holds the anchoring element in a straightened configuration. A further step 230 of method 200 may be moving the rigid elongated reinforcing element to release the anchoring element to the pre-biased deflected form. A further step may involve pushing the rigid elongated reinforcing element, which may be a rod, axially along the channel 32 so as to deflect the segments (areas of the screw 10 between successive cut-outs 33) back to the straight configurations.

Method 200 may in some preferred embodiments involve moving the rigid elongated reinforcing element through a channel that is on a surface of the shaft, for example as shown in FIG. 11A and FIG. 11B. Method 200 may in other preferred embodiments involve moving the rigid elongated reinforcing element through a channel that is situated centrally with respect to a dimension on a plane perpendicular to the longitudinal axis, as shown in FIG. 12A and FIG. 128. The method 200 may alternatively involve moving the rigid elongated reinforcing element through a channel that is entirely within the core.

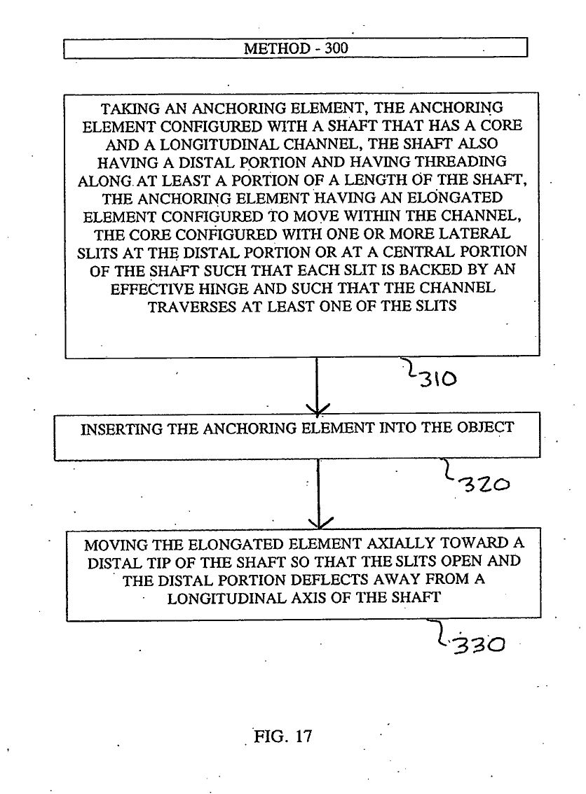

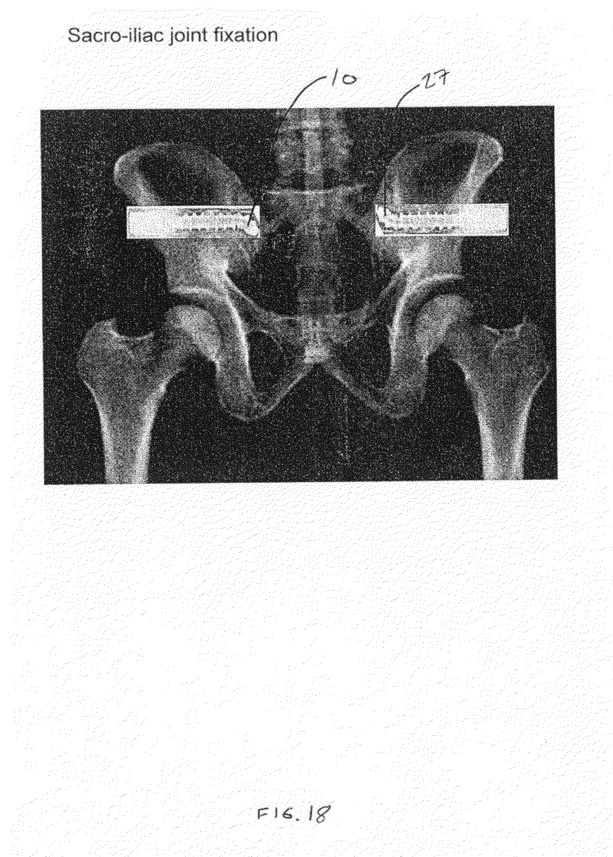

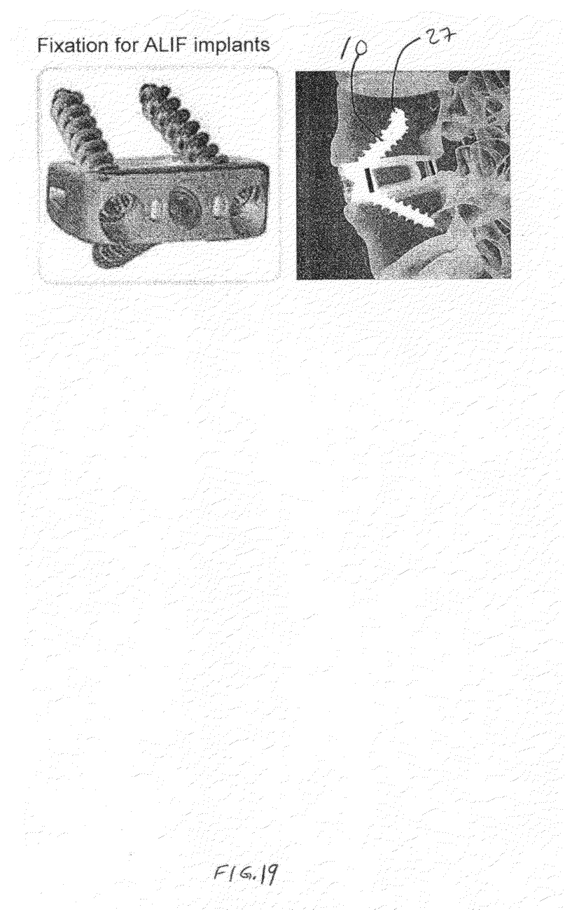

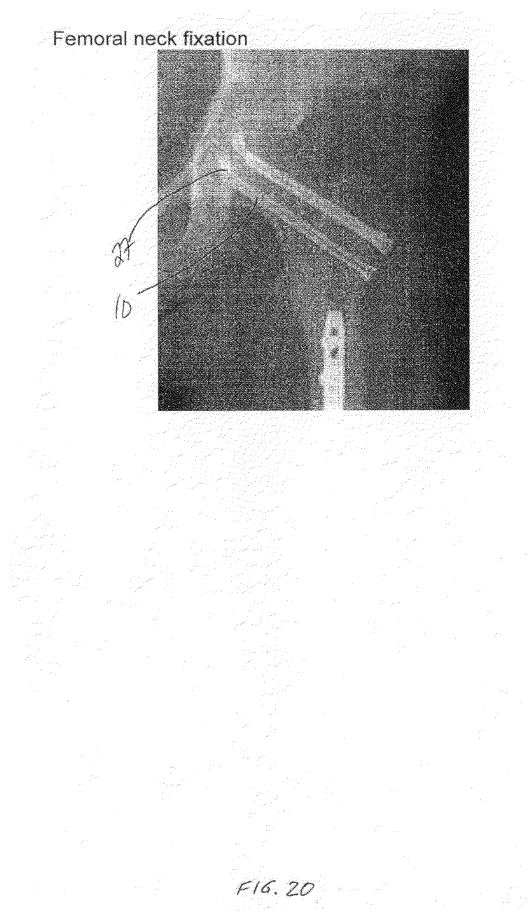

As shown in FIG. 17, the present invention may be described as a method 300 of inserting an anchoring element into an object. Step 310 of the method 300 may comprise taking or employing an anchoring element, the anchoring element configured with a shaft that has a core and a longitudinal channel, the shaft also having a distal portion and having threading along at least a portion of a length of the shaft, the anchoring element having an elongated element configured to move within the channel, the core configured with one or more lateral slits (see FIG. 15AA) at the distal portion or at a central portion of the shaft such that each lateral slit is backed by an effective hinge and such that the channel traverses at least one of the slits.

A further step 320 may comprise inserting the anchoring element into the object. Step 330 may be moving (i.e. pushing) the elongated element 40a axially toward a distal tip 27a of the shaft so that the slits open (see FIG. 158) and the distal portion deflects away from a longitudinal axis of the shaft. As shown in FIGS. 15AA and FIG. 15B, method 300 may further comprise using an elongated element that is flexible enough to bend during deflection but rigid enough to effectuate deflection of the distal portion. Steps 320-330 of method 300 may be accomplished by inserting the anchoring element into the object with the elongated element 40a already in the channel 32 of anchoring element 10 and then exerting an axial pushing force on elongated element 40a in step 330. Alternatively, steps 320-330 may be accomplished by inserting anchoring element 10 into the object without elongated element 40a being in the channel 32 in step 320 and then in step 330 inserting elongated element 40a as far as it can go in channel 32 and further pushing the elongated element 40a further by exerting an axial pushing force to cause the deflection.

Although illustrated herein in a non-limiting preferred embodiment where the deflected portion is substantially at the distal tip of the screw, i.e., with the deflection beginning at a distance of no more than two core diameters from the tip, alternative embodiments in which flexing occurs at any other location along the screw (and with any number of hinges/joints) also fall within the scope of the present invention.

It should also be noted that the invention is not limited to simple forms of deflection as shown, and could alternatively be implemented using non-parallel effective hinges formed by cut-outs at different angles around the axis, thereby achieving helical or other geometrical forms.

The core of the screw may have a conical shape or a cylindrical shape. The thread projecting from the core of the screw may follow the shape of the core, so that the screw itself may assume a cylindrical or conical shape, or may have an external shape (the envelope of the outer edges of the thread) which differs from the shape of the core to provide any desired combination of thread and core shapes.

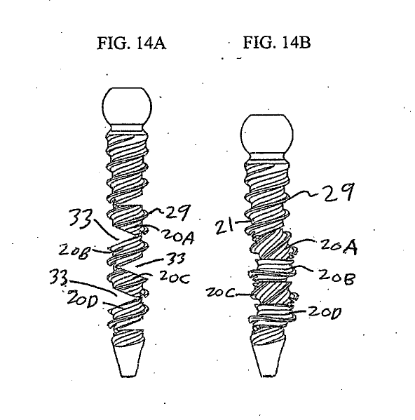

Finally, as illustrated in FIGS. 7A and 7B, the invention is not limited to deflection in a single direction. In an alternative embodiment, the distal portion 27 of the screw (which for example may be the last 10%, the last 20%, the last 30%, the last 40%, the last 50% or some other last portion of the screw or anchoring element 10, in various different preferred embodiments) may be subdivided into two or more elongated segments 93A, 93B which are selectively deflected in different directions. The underlying principles of structure and operation remain the same as described above. In the case illustrated here of two elongated portions deflected in opposite directions, the inwardly facing flat surfaces become the flexible backbones providing the effective hinges or joints between segments of the two halves, without the need for relief slits opposite each V-shaped cut-out. Accordingly, the previously mentioned statement that the distal portion 27 of the shaft 20 may deflect to “an angle” from the longitudinal axis of the shaft should be understood to also refer a case such as in FIG. 7B where the distal portion 27 has two segments that each deflect to an angle but to opposite angles (from the longitudinal axis 25 of the shaft 20) and also even to a case where the two segments deflect to different angles (from the longitudinal axis 25). Each segment 93A, 93B taken alone can also be considered a distal portion 27 that deflects to an angle from the longitudinal axis. FIGS. 14A-14B show a preferred embodiment in which deflection occurs in alternating directions. For example, a first deflected segment 20A may deflect in a first direction, a second deflected segment 20B may deflect in a second direction, a third deflected segment 20C may deflect in the first direction and a fourth deflected segment 20D may deflect in the second direction. The segments are defined as the portion of the screw between consecutive cut-outs 33. Another may of describing the screw 10 of FIGS. 14A-B is to state it has cut-outs 33 that appear in alternate directions along shaft 20, and in this case on generally opposing sides of shaft 20.

In all cases, the bone screw of the present invention may be made from any biocompatible material with suitable mechanical properties, and sufficiently flexible or deformable to provide the required deflection. Finally, it is noted that the channel 32 of an anchoring element 10, for example a bone screw, may also be used to inject any biocompatible material, such as bone cement, into the tissue where the anchoring element 10 is inserted, for example the vertebra, of a subject.

The invention has been exemplified thus far in a particularly preferred set of embodiments as a bone screw. It should be noted however that the structure as defined herein may be used to advantage in a range of other human and non-human medical applications. Furthermore, the structure finds valuable applications in non-medical mechanical application.

Although described herein with reference to screws, it should be noted that some aspects of the present invention may be implemented to advantage in non-threaded anchoring elements, such as nails or rods which are driven into or otherwise inserted into bone (or some other body or object) in a straight state and subsequently some part of the distal half of the device is made to deflect in order to provide enhanced pullout resistance and/or resistance to loosening. The devices may have round, square or other cross-sectional shapes. The structure, function and advantages of such devices will be understood by analogy to the screw embodiments discussed above.

As used herein, the term “about” in reference to a quantitative amount means plus or minus five percent. Substantially parallel lines or axes are completely parallel or intersect (or would intersect if extended) at an angle of 10 rotational degrees or less.

While the invention has been described with respect to a limited number of embodiments, it will be appreciated that many variations, modifications and other applications of the invention may be made. Therefore, the claimed invention as recited in the claims that follow is not limited to the embodiments described herein.

1. An anchoring element, comprising:
a shaft having a core and a longitudinal channel, the shaft also having a distal portion and having threading along at least a portion of a length of the shaft;
an elongated element movable within the channel, the elongated element being (i) a tensioning element or (ii) a rigid element or (iii) a tensioning element and a rigid element;
one or more lateral cut-outs in the core at the distal portion or at a central portion of the shaft, each cut-out backed by an effective hinge, the channel traversing at least one side of one of the cut-outs,
the elongated element and distal portion configured such that upon axial movement of the elongated element, the distal portion deflects to an angle from a longitudinal axis of the shaft.

2. The anchoring element of claim 1, wherein the elongated element is a tensioning element attached to the distal portion such that the axial movement pulls the distal portion to deflect the distal portion.

3. The anchoring element of claim 2, wherein the tensioning element is situated at a distance from the longitudinal axis.

4. The anchoring element of claim 2, wherein the axial movement of the elongated element is toward a proximal end of the shaft.

5. The anchoring element of claim 2, wherein the axial movement of the elongated element is toward a distal tip of the shaft.

6. The anchoring element of claim 1, wherein the elongated element is a rigid element and the anchoring element is pre-biased to assume a deflected form such that the distal portion is at an angle to the longitudinal axis and the rigid element maintains the anchoring element in a straight configuration until the axial movement of the rigid element.

7. The anchoring element of claim 6, wherein the channel is on a surface of the shaft.

8. The anchoring element of claim 6, wherein the channel is situated centrally with respect to a dimension on a plane perpendicular to the longitudinal axis.

9. The anchoring element of claim 1, wherein the channel is entirely inside the core.

10. The anchoring element of claim 1, wherein at least one effective hinge is an integral hinge.

11. The anchoring element of claim 1, further comprising a transverse relief slit between at least one integral hinge and a surface of the shaft opposite a cut-out backed by the at least one integral hinge.

12. The anchoring element of claim 1, wherein each integral hinge is displaced from the longitudinal axis.

13. The anchoring element of claim 1, wherein the cut-outs are V-shaped in cross-section.

14. The anchoring element of claim 1, wherein the cut-outs are symmetrical about a plane that is at 90° to the longitudinal axis.

15. The anchoring element of claim 1, wherein the cut-outs are proximal to a distal tip.

16. The anchoring element of claim 1, wherein the cut-outs interrupt the threading around the shaft.

17. The anchoring element of claim 1, wherein the angle is at least 10 degrees.

18. The anchoring element of claim 1, wherein the angle is between about 10 degrees and about 180 degrees.

19. The anchoring element of claim 1, wherein the angle is between about 70 and about 110 degrees.

20. The anchoring element of claim 1, wherein the core has a conical shape or a cylindrical shape.

21. The anchoring element of claim 1, further comprising a tulip-shaped hollow connector on a proximal end of the shaft for accommodating a rod connecting two or more pedicle screws.

22. The anchoring element of claim 1, further comprising a locking mechanism for maintaining deflection of the distal portion.

23. The anchoring element of claim 1, wherein the longitudinal channel is not straight prior to insertion of the elongated element and wherein the elongated element and channel are configured so that pushing the elongated element through the longitudinal channel toward a distal tip deflects the distal portion by putting the longitudinal channel into a straight configuration from a non-straight configuration.

24. A method of inserting an anchoring element into an object, comprising:
taking an anchoring element, the anchoring element configured with a shaft that has a core and a longitudinal channel, the shaft also having a distal portion and having threading along at least a portion of a length of the shaft, the anchoring element having an elongated element configured to move within the channel, the core configured with one or more lateral cut-outs at the distal portion or at a central portion of the shaft such that each cut-out is backed by an effective hinge and such that the channel traverses at least one side of one of the cut-outs,
inserting the anchoring element into the object; and
moving the elongated element axially so that the distal portion deflects away from a longitudinal axis of the shaft.

25. The method of claim 24, further comprising moving the elongated element axially toward a proximal end of the shaft.

26. The method of claim 24, further comprising moving the elongated element axially toward a distal tip of the shaft.

27. The method of claim 24, further comprising the distal portion deflecting to an angle from the longitudinal axis of between about 10 degrees and about 180 degrees.

28. The method of claim 24, further comprising distal portion deflecting to an angle from the longitudinal axis of between about 70 and about 110 degrees.

29. The method of claim 24, further comprising reducing axial loading on the distal portion by having the deflection of the distal portion create a region of very low bone density in the object adjacent the deflected distal portion.

30. The method of claim 29, further comprising creating an area of increased concentration in the object adjacent the deflected distal portion on an opposite side of the distal portion from the region of very low bone density.

31. The method of claim 24, further comprising deflecting the distal portion until the cut-outs close together.

32. The method of claim 24, further comprising taking an anchoring element whose core is configured with a transverse slit on a surface of the core opposite a cut-out, for at least one cut-out, to facilitate deflection of the anchoring element.

33. The method of claim 24, further comprising straightening the deflected distal portion by pushing the elongated element axially in a distal direction until the anchoring element returns to an original straight configuration.

34. The method of claim 24, further comprising taking an anchoring element whose channel is displaced from the longitudinal axis.

35. A method of inserting and removing an anchoring element into and from an object, comprising:
taking an anchoring element in accordance with claim 24;
straightening the deflected distal portion by pushing the elongated element axially in a distal direction until the anchoring element returns to an original straight configuration; and
removing the anchoring element from the object.

36. The method of claim 24, further comprising moving the elongated element axially so that the distal portion deflects away from a longitudinal axis of the shaft is effectuated by pushing the elongated element through a longitudinal channel to put the longitudinal channel into a straight configuration from a non-straight configuration.

37. A method of inserting an anchoring element into an object, comprising:
taking an anchoring element that is pre-biased to assume a deflected form such that a distal portion of a shaft of the anchoring element is at an angle to a longitudinal axis of the shaft, the shaft also having threading along at least a portion of a length of the shaft, the anchoring element configuring with a longitudinal channel housing a rigid elongated reinforcing element configured to move within the channel, the core having one or more lateral cut-outs at the distal portion or at a central portion of the shaft, each cut-put backed by an effective hinge, the channel traversing at least one side of one of the cut-outs;
inserting the anchoring element into the object with the longitudinal channel housing a rigid elongated reinforcing element that temporarily holds the anchoring element in a straightened configuration; and

38. The method of claim 37, further comprising moving the rigid elongated reinforcing element through a channel that is on a surface of the shaft

39. The method element of claim 38, further comprising moving the rigid elongated reinforcing element through a channel that is situated centrally with respect to a dimension on a plane perpendicular to the longitudinal axis.

40. The method element of claim 37, further comprising moving the rigid elongated reinforcing element through a channel that is entirely within the core.

41. The method of claim 37, further comprising moving the rigid elongated reinforcing element axially through the channel toward a proximal end of the shaft.

42. The method of claim 37, further comprising moving the rigid elongated reinforcing element axially through the channel toward a distal tip of the shaft.

43. A method of inserting an anchoring element into an object, comprising:
taking an anchoring element, the anchoring element configured with a shaft that has a core and a longitudinal channel, the shaft also having a distal portion and having threading along at least a portion of a length of the shaft, the anchoring element having an elongated element configured to move within the channel, the core configured with one or more lateral slits at the distal portion or at a central portion of the shaft such that each lateral slit is backed by an effective hinge and such that the channel traverses at least one of the slits,
inserting the anchoring element into the object; and
moving the elongated element axially toward a distal tip of the shaft so that the slits open and the distal portion deflects away from a longitudinal axis of the shaft.

44. The method of claim 43, further comprising using an elongated element that is flexible enough to bend during deflection but rigid enough to effectuate deflection of the distal portion.

45. An anchoring element, comprising:
a shaft having a core and a longitudinal channel, the shaft also having a distal portion and having threading along at least a portion of a length of the shaft;
an elongated element movable within the channel, the elongated element being flexible enough to bend during deflection but sufficiently rigid to effectuate the deflection;
one or more lateral cut-outs in the core at the distal portion or at a central portion of the shaft, each cut-out backed by an effective hinge, the channel traversing at least one side of one of the cut-outs,
the elongated element and distal portion configured such that upon axial movement of the elongated element, the distal portion deflects to an angle from a longitudinal axis of the shaft.
